(19) United States
(12) Patent Application Publication
(10) Pub. No.: US 2023/0010686 A1
(43) Pub. Date: Jan. 12, 2023

(54) GENERATING SYNTHETIC PATIENT HEALTH DATA

(71) Applicant: The Regents of the University of California, Oakland, CA (US)

(72) Inventors: Michael D. Lesh, San Francisco, CA (US); Ofer Mendelevitch, San Francisco, CA (US); Gil Tamari, San Francisco, CA (US)

(21) Appl. No.: 17/782,551

(22) PCT Filed: Dec. 4, 2020
(86) PCT No.: PCT/US20/63433

§ 371 Date: Jun. 3, 2022

Related U.S. Application Data
(60) Provisional application No. 62/944,317, filed on Dec. 5, 2019.

(51) Int. Cl. G16H 10/60; G06N 3/08

(52) U.S. Cl. CPC G16H 10/60; G06N 3/08

(57) ABSTRACT

Systems and methods for generating synthetic medical data are provided. A method may include retrieving a set of authentic electronic medical records from a database. The method may further include converting the authentic set of electronic medical records to a set of numerical vectors. The method may further include training a first neural network based on a random noise generator sample, the first neural network outputting synthetic electronic medical records. The method may further include training a second neural network based on the output synthetic electronic medical records and the set of numerical vectors, the second neural network outputting a loss distribution indicating whether the output synthetic electronic medical records are classified as authentic or synthetic.

### CROSS-REFERENCES TO RELATED APPLICATIONS

This application claims the benefit of U.S. Provisional Application No. 62/944,317, filed Dec. 5, 2019, which is incorporated herein by reference in its entirety and for all purposes.

### TECHNICAL FIELD

The subject matter described herein relates generally to machine learning and more specifically to generating synthetic patient health data by a machine learning model.

### BACKGROUND

Machine learning models may be trained to perform a variety of cognitive tasks including, for example, object identification, natural language processing, information retrieval, speech recognition, classification, regression, and/or the like. For example, an enterprise resource planning (ERP) system may include an issue tracking system configured to generate a ticket in response to an error reported via one or more telephone calls, emails, short messaging service (SMS) messages, social media posts, web chats, and/or the like. The issue tracking system may generate the ticket to include an image or a textual description of the error associated with the ticket. As such, in order to determine a suitable response for addressing the error associated with the ticket, the enterprise resource planning system may include a machine learning model trained to perform text or image classification. For instance, the machine learning model may be trained to determine, based at least on the textual description of the error, a priority for the ticket corresponding to a severity of the error.

### SUMMARY

Systems, methods, and articles of manufacture, including computer program products, are provided for preparing data for machine learning processing and synthetic data generation. In one aspect, there is provided a system including at least one data processor and at least one memory. The at least one memory may store instructions that cause operations when executed by the at least one data processor. The operations may include retrieving a set of authentic electronic medical records from a database. The operations may further include converting the authentic set of electronic medical records to a set of numerical vectors. The operations may further include training a first neural network based on a random noise generator sample, the first neural network outputting synthetic electronic medical records. The operations may further include training a second neural network based on the output synthetic electronic medical records and the set of numerical vectors. The second neural network outputting a loss distribution indicating whether the output synthetic electronic medical records are classified as authentic or synthetic. Training the first neural network further includes updating a first gradient of the first neural network based on the loss distribution. Training the second neural network further includes updating a second gradient of the second neural network based on the loss distribution.

In some variations, one or more features disclosed herein including the following features can optionally be included in any feasible combination. Training the first neural network may further include receiving a conditioning modifier. The conditioning modifier may alter at least one characteristic of the synthetic electronic medical records. The conditioning modifier may be received via a user interface. Training the first neural network may be in response to receiving a request for synthetic electronic medical records from a front end system. Updating the first gradient may include descending the first gradient. Updating the second gradient may include ascending the second gradient. The first neural network may include a recurrent neural network. The recurrent neural network may utilize a time aware long short-term memory. The recurrent neural network may utilize a gated recurrent new unit. The operations may further include validating the synthetic medical records. The validating may include comparing a statistical distribution of the synthetic medical records to a statistical distribution of the authentic medical records. The validating may further include comparing a predictive model performance of the synthetic medical records to a predictive model performance of the authentic medical records. The second neural network may be distributed across multiple devices in separate locations in a federated learning structure.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to preparing data for machine learning processing, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating a data processing system, in accordance with some example embodiments;

FIG. 2 is a block diagram of a synthetic patient health data generator, in accordance with some example embodiments;

FIG. 3 depicts a system diagram illustrating an example system for generating synthetic patient health data, in accordance with some example embodiments;

FIG. 4 depicts a diagram illustrating an example user interface system for generating synthetic patient health data, in accordance with some example embodiments;

FIG. 5A depicts an example embedding vector generation process, in accordance with some example embodiments;

FIG. 5B depicts an example embedding process for generating numerical vectors from electronic medical records, in accordance with some example embodiments;

FIG. 6 depicts training an example generative adversarial network, in accordance with some example embodiments;

FIG. 7 depicts training an example generative adversarial network using federated learning, in accordance with some example embodiments;

FIG. 8 depicts a block diagram illustrating a computing system, in accordance with some example embodiments; and

FIG. 9 depicts a flowchart illustrating an example of a process for generating synthetic patient data, in accordance with some example embodiments.

When practical, similar reference numbers denote similar structures, features, or elements.

### DETAILED DESCRIPTION

The adoption of electronic health records (EHR) by healthcare organizations has led to an increase in medical data available, as well as the number of applications of machine learning and AI that utilize such “big data.”

However, the wide adoption of electronic health record systems does not automatically lead to easy access to electronic health record data for academic or industry researchers. There is a wide concern around sharing such data, primarily due to patient privacy concerns. Thus, usage of electronic health record data in research settings is limited due to this regulation and internal controls that are implemented by healthcare organizations to protect against misuse or data breaches.

There have been various approaches proposed to address this issue, and enable broader usage of electronic health record data in research, including data de-identification; however, none of these solutions are deemed satisfactory at this point; some are not scalable enough, while others are considered vulnerable to various security threats and attacks.

De-identification, the process of anonymizing datasets before sharing them, has been the main paradigm used in research and elsewhere to share data while preserving individual privacy. Until recently, data protection laws worldwide consider anonymous data as not personal data anymore, allowing it to be freely used, shared, and sold. Academic journals are increasingly requiring authors to make anonymous data available to the research community. However, while standards for anonymous data vary, many data protection laws consider that each and every person in a dataset has to be protected for the dataset to be considered anonymous.

A recent quantitative analysis of the risks associated with de-identification on 210 different populations, showed that 99.98% of Americans would be correctly re-identified in any dataset using 15 demographic attributes. The results of the analysis suggest that even heavily sampled anonymized datasets may be unlikely to satisfy at least some standards for anonymization and seriously challenge a technical and/or a legal adequacy of the de-identification release-and-forget model.

This disclosure describes a method and system to generate synthetic but realistic electronic health record data, utilizing state-of-the-art techniques in deep machine learning, generative models, reinforcement learning and federated learning to provide a robust and realistic synthetic electronic health record dataset. Access by researchers or other third parties to synthetic data described herein is intended not to violate privacy of the underlying authentic patients.

Usage of such synthetic data may be as a stand-alone electronic health record dataset for various healthcare applications utilizing predictive models or as groups with which to make comparisons, such as a synthetic control group, as well as a way to complement or augment existing electronic health record datasets to achieve better outcomes. Furthermore, using conditioning of the generative models (e.g., cGAN) may allow the system to alter the statistical characteristics of the generated dataset towards various applications, such as dealing with rare conditions.

A method of obtaining new medical insights may be through empirical clinical research. Unfortunately, in medicine the ability to conduct clinical research is severely limited by the high cost of enrolling and following patients, the long follow-up times, the large number of options to be compared, the large number of patients, unwillingness of people to participate (e.g., to be randomized or to follow a specified protocol), and unwillingness of the world to stand still until the research is done. A typical clinical trial comparing just two pharmacologic options requires thousands of patients, costs tens or hundreds of millions of dollars, may take 3 to 15 years, and is likely to be outdated before it is completed.

Access to data may be essential for research, and for training machine learning (ML) models. However, obtaining real-world data, especially the massive quantities required for machine learning, may be costly and often present legal and privacy concerns. This may be particularly challenging in healthcare, where health records may contain highly sensitive information and may be strictly protected by privacy laws such as the health insurance portability and accountability act of 1996 (HIPAA) in the US and the general data protection regulation 2016/679 (GDPR) in Europe, and various other organizational policies.

To circumvent these challenges, and given that various de-identification algorithms fail to prevent re-identification, some have developed approaches to synthesize clinical data. However, in the majority of methods, rules (such as practice guidelines, those derived from the medical literature, etc.) are used to construct a synthetic data stream that is relatively coarse-grained and by definition lacks the inherent complexity of real data. Utilizing deep learning techniques may enable the generated synthetic data to more truly capture nuanced patterns in the actual patient records, as opposed to rules-based methods which can only generate patterns that were specifically programmed into the rules by domain experts. Using machine learning means that extant patterns, of which medical experts or other sources of rules are not yet aware, may be captured in a deep machine learning method.

Rule-based approaches are derived from some probability-based logic and completely bypass the use of real patient-level data. The benefit of rule-based synthesis is that they may pose little risk for revealing personally identifiable information. However, the rule-based synthetic data may be limited in terms of features (data points) and the quantity of patient records synthesized. A rules-based synthesis engine cannot use conditioning to alter the characteristics of the database (e.g., incidence of a given diagnosis or genetic marker), nor is a deep learning method employed. The purpose of the synthetic version of the database is simply to allow research queries to be done on a limited quantity of real data in a way that bypasses privacy issues. And the more specific the query population, the more limited are the questions that can be asked of the database. As such, the synthetic data using a rule-based approach cannot be used to train a machine learning model.

The methods for generating synthetic patient health data described herein may not have such limitations and may not have privacy issues irrespective of the number of patient records created or underlying incidents of a given patient characteristic such as diagnosis. For example, the synthetic patient health information generated using the processes described herein may have an output that may be purely synthetic and may be mathematically shown to not be re-identifiable. Additionally, the synthetic patient health generation method described herein may beneficially have no limit on datatypes as input to a synthetic generator previous technology only allowed either categorical or continuous input to the generative model.

The system and methods described herein may be used to generate a synthetic electronic health record dataset or augment an existing electronic health record dataset to make it more usable for downstream applications. Augmentation herein may refer to adding additional patient records of an existing electronic health record dataset or to extend and enhance existing records with more data.

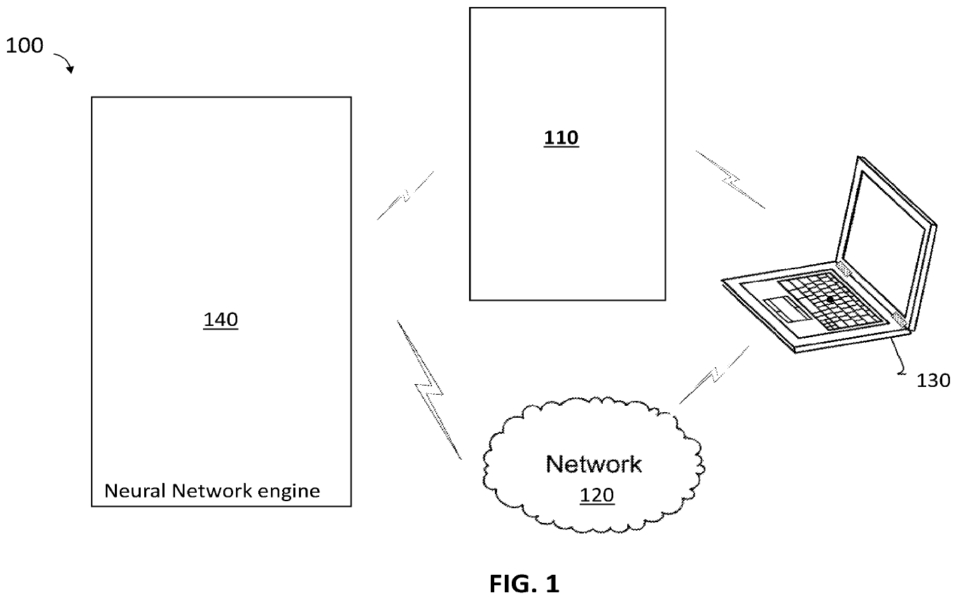

FIG. 1 depicts a network diagram illustrating a network environment 100, in accordance with some example embodiments. Referring to FIG. 1, a training engine 110 may be communicatively coupled, via a wired and/or wireless network 120, to client device 130 and/or a neural network engine 140. The wired and/or wireless network 120 can be a wide area network (WAN), a local area network (LAN), and/or the Internet.

In some example embodiments, the neural network engine 140 may be configured to implement one or more machine learning models including, for example, a recurrent neural network. A recurrent neural network is a class of artificial neural networks where connections between nodes form a directed graph along a temporal sequence. A recurrent neural network may use their internal state (memory) to process sequences of inputs. As such, the neural network engine 140 may be trained to serve as, for example, an image or data generator and/or classifier. According to some example embodiments, the training engine 110 may be configured to generate a mixed training set that includes both synthetic data and non-synthetic data. The training engine 110 may be further configured to process the mixed training set with a recurrent neural network (e.g., implemented by the neural network engine 140) and determine the performance of the neural network in classifying the data included the mixed training set. According to some example embodiments, the training engine 110 may generate, based at least on the performance of the recurrent neural network, additional training data. The additional training data may include data with modifications that may cause the recurrent neural network to misclassify one or more synthetic data in the mixed training set.

In some example embodiments, the training engine 110 may generate synthetic data (e.g., synthetic patient medical records) based on non-synthetic data (e.g., authentic historical patient medical records) that are associated with one or more labels. For instance, a non-synthetic data may depict a patient health record having one or more medical events. The labels associated with the non-synthetic data may correspond to the medical events. To generate the synthetic data, the training engine 110 may apply, to the non-synthetic data, modifications to portions of the non-synthetic data. For example, the non-synthetic image may be modified by, for example, modifying the patient information and/or modifying the medical events. The quantity of non-synthetic data may be substantially lower than the quantity of synthetic data that may be generated based on the non-synthetic data.

In some example embodiments, the client device 130 may provide a user interface for interacting with the training engine 110 and/or neural network engine 140. For example, a user may provide, via the client device 130, at least a portion of the non-synthetic data used to generate the mixed training set. The user may also provide, via the client device 130, one or more training sets, validation sets, and/or production sets for processing by the neural network engine 140. Alternately and/or additionally, the user may provide, via the client device 130, one or more configurations for the neural network engine 140 including, for example, conditional parameters (e.g., modifiers) such as demographic/statistical information or characteristics (e.g., race, age, genetic marker, disease, or the like) that is used by the neural network engine 140 when processing one or more mixed training sets, validation sets, and/or production sets. The user may further receive, via the client device 130, outputs from the neural network engine 140 including, for example, classifications for the mixed training set, validation set, and/or production set.

In some example embodiments, the functionalities of the training engine 110 and/or the neural network engine 140 may be accessed (e.g., by the client device 130) as a remote service (e.g., a cloud application) via the network 120. For instance, the training engine 110 and/or the neural network engine 140 may be deployed at one or more separate remote platforms. Alternately and/or additionally, the training engine 110 and/or the neural network engine 140 may be deployed (e.g., at the client device 130) as computer software and/or dedicated circuitry (e.g., application specific integrated circuits (ASICs)).

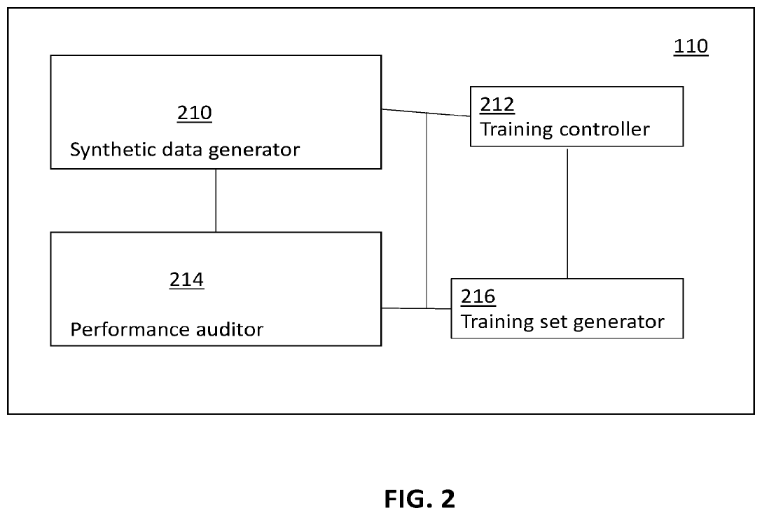

FIG. 2 depicts a block diagram illustrating the training engine 110, in accordance with some example embodiments. Referring to FIGS. 1-2, the training engine 110 may include a synthetic data generator 210, a training controller 212, a performance auditor 214, and a training set generator 216. It should be appreciated that the training engine 110 may include additional and/or different components.

As noted above, the training engine 110 may be configured to generate a mixed training set for training a neural network (e.g., implemented by the neural network engine 140). In some example embodiments, the synthetic data generator 210 may be configured to generate a plurality of synthetic electronic health records that are included in a mixed training set used for training the neural network. The synthetic data generator 210 may generate one or more synthetic electronic health records by at least generating the synthetic electronic health records based on a random noise generator.

The electronic health record data may contain multiple patient records, each including of one or more medical events recorded during patient care. Since multiple events may be created per synthetic patient, the synthetic data may be longitudinal, that is, not a set of static characteristics of a patient such as age, gender, diagnoses, but a complete patient trajectory of medical events over time that can include multiple physician contacts, lab tests, hospital admissions, surgeries, etc. Synthetic data may also include synthetic unstructured data such as physician notes, created via natural language generators.

In some example embodiments, the training controller 212 may conduct additional training of the neural network based at least on the performance of the neural network in processing a mixed training set (e.g., as determined by the performance auditor 214). The training controller 212 may train the neural network using additional training data that have been generated (e.g., by the synthetic image generator 210 and/or the training set generator 216) to include synthetic electronic health records that have been subject to modifications that the performance auditor 214 determines to cause the neural network to misclassify synthetic data. Referring to the previous example, the performance auditor 214 may determine that the neural network is unable to successfully classify, for example, a threshold quantity (e.g., number, percentage) of synthetic electronic health records from authentic electronic health records. As such, the synthetic data generator 210 may generate additional synthetic electronic health records having changed characteristics.

Meanwhile the training controller 212 may train the neural network with additional training data that includes the synthetic electronic health records with changed characteristics (e.g., generated by the synthetic data generator 210). The training controller 212 may continue to train the neural network with additional training data until the performance of the neural network (e.g., as determined by the performance auditor 214) meets a certain threshold value (e.g., fewer than x number of misclassifications per training set and/or validation set) or a loss distribution determined by the neural network satisfies a threshold value.

In some example embodiments, the performance auditor 214 may be configured to determine the performance of a neural network (e.g., implemented by the neural network engine 140) in processing the mixed training set. For example, the performance auditor 214 may determine, based on a result of the processing of a mixed training set performed by the neural network, that the neural network misclassifies synthetic electronic health records from the mixed training set that have been subject to certain modifications. To illustrate, the performance auditor 214 may determine, based on the result of the processing of the mixed training set, that the neural network (e.g., a discriminator) misclassified, for example, a first synthetic electronic health record. The first synthetic electronic health record may be generated by at least the synthetic data generator 210 generating the first synthetic electronic health record based on a random noise generated training dataset. Accordingly, the performance auditor 214 may determine that the neural network (e.g., a discriminator) may be unable to successfully classify synthetic electronic health record from non-synthetic electronic health records. The performance auditor 214 may include a discriminator model that is updated with new synthetic electronic health records or a loss distribution generated from the discriminator model to improve its ability to discriminate between synthetic and non-synthetic electronic health records.

In some example embodiments, the training set generator 216 may generate a mixed training set for training a neural network (e.g., implemented by the neural network engine 140). The mixed training set may include non-synthetic data, e.g., authentic electronic health records. The training set generator 216 may obtain the mixed training set from the client device 130.

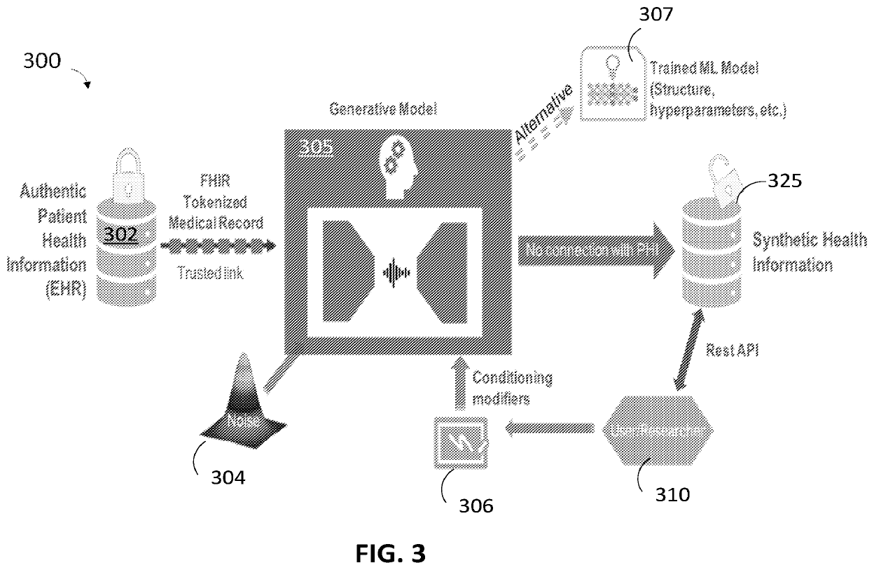

FIG. 3 depicts a system diagram illustrating an example system 300 for generating synthetic patient health data, in accordance with some example embodiments. As shown in the example of FIG. 3, data from an authentic health information (e.g., non-synthetic electronic health record data) source or storage 302 may be provided as input to a generative model 305, and used to train this model to generate synthetic electronic health records. The generative model may include a statistical model used to generate random instances (e.g., electronic health records), either of an observation and target, or of an observation x given a target value y. For example, the data may include electronic health records from a hospital or other medical facility. This data may be in any format, such as a fast healthcare interoperability resource (FHIR) format. The electronic health record data may contain multiple patient records, including one or more medical events recorded during patient care.

Additionally, noise 304 may also be provided as input to the generative model 305. The generative model 305 may also receive conditioning modifiers 306 as input to train the model 305. The conditioning modifiers 306 may be inputted by an end user 310 using an interface (e.g., a REST API). A user, using a representational state transfer (REST) application programming interface (API) or a graphical user-interface, may define a set of conditioning modifiers (also known as conditioning parameters) that may determine desired characteristics and a probability density function of the synthetic electronic health record data, and may control what will be included in the output, as well as various statistical characteristics of the output.

The conditioning modifiers may represent a set of user-defined parameters that are provided (by the user) as input to the system 300 in order to influence and/or modify a characteristic of the synthetic electronic health record data 325 generated, so that it is biased towards an outcome of choice. For example, a modifier might be used to generate synthetic electronic health records with a certain distribution of ethnic groups or an increase in a percentage of a given diagnosis or a genetic marker.

An aspect of the system 300 is the complete separation between the source (authentic) electronic health record data, which may be secure and remain at rest and may be used only for training the generative model, and the output data which is synthetic (i.e., likely to not contain any real patient data nor any other patient identifying information). In some implementations, the system 300 may include multiple sources of authentic electronic health record data that can then be combined directly or using federated learning techniques, allowing the system to perform learning (e.g., training generative models) without co-locating any parts of the electronic health record data in a centralized location. That is, the system 300 may allow synthetic data to be generated from a single or multiple source databases 302 at rest, with no requirement that the source data 302 be moved or copied to an alternative location. The source data may be physically located at a hospital site or in a cloud storage site such as Amazon Web Service or Google Cloud Platform.

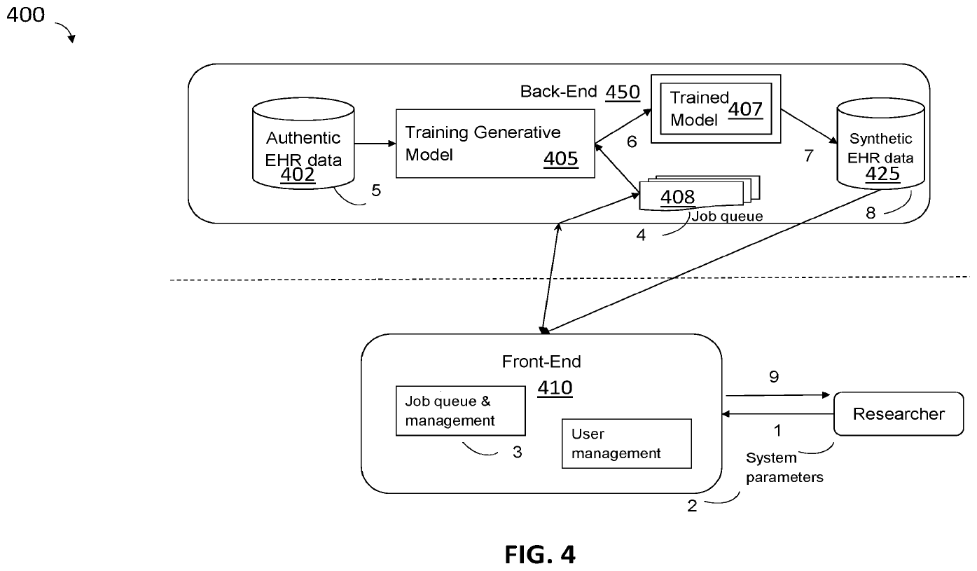

FIG. 4 depicts a diagram illustrating an example user interface system 400 for generating synthetic patient health data, in accordance with some example embodiments. The system 400 may include a front end system 410, and a back-end system 450. The back end system 450 may include an authentic electronic health record database 402, a generative model 405, the trained generative model 407, a job queue 408 and a synthetic electronic health record database 425. In some aspects, the back end system 450 may be implemented using a container technology such as Docker, Linux, Window, a database container, or the like.

In the example of FIG. 4, at 1, an end-user (e.g., a researcher) may login to the front end system 410. Credentials of the end-user may be validated against a user role or policy, which may govern what types of activities the end-user may perform on the system 400 and/or what types of synthetic data the end-user may generate.

At 2, the end-user may define (via a graphical user interface) parameters that may govern the process of synthetic data generation, such as a quantity of records to generate, a time period (e.g., a start date and an end date) for medical records and the synthetic data set, a time related granularity of generated records (e.g., hourly, daily, or by clinic visit or hospital admission, or the like), conditioning modifiers that may control the synthetic data generated, or the like.

At 3, the front end system 410 may create a “job object” (e.g., in JSON format) that may encapsulate the various parameters and associated metadata that may be required to generate synthetic data (e.g., synthetic electronic health records). The front end system 410 may send the job object to the back-end system 450.

At 4, the back end system 450 may add the received job object to the job queue 408. The job queue 408 may include a list of scheduled jobs and may create an audit log entry for the job received from the front end system 410.

At 5, jobs in the job queue 408 may be executed based on a queuing priority mechanism. On a job execution, the back end system 450 may access authentic electronic health records from the database 402 and use the authentic data to train the generative model 405 based at least in part on the job object definitions and parameters which may have been defined in the front end system 410. The output of the training process may be the trained model 407 which may be stored in the backend system 450 and tagged appropriately for future retrieval/use. In some aspects, the trained model 407 may be further used as a starting point for other models using transfer learning techniques.

At 6, after training of the generative model 405 is finished, a synthetic electronic health record dataset (a set of synthetic electronic health records in a format such as FHIR format) may be generated using the trained model 407 and stored in the database 425 of the backend system 450.

At 7, the back end system 450 may run a data validation process that includes various quality control mechanisms as well as validation that the generated synthetic data set is compatible with expected statistical distributions as may be defined in the generation parameters. In some aspects, an electronic watermark may be added to an image, audio, video, or other data items where watermarking is applicable.

At 8, the back-end system 450 may copy the generated synthetic dataset to the front end system 410.

At 9, once the synthetic data is available in the front end system 410, the end user may be notified of its availability and may request access to their data in a variety of ways such as retrieving a complete copy of the data set, querying the data and retrieving a subset of the data set (e.g., getting a smaller subset of the patients), running an analytic or machine learning task on the front end system 410 that may utilize the synthetic dataset, or the like. Each access to the synthetic data by the end-user may be recorded in the audit log.

As noted above, electronic health record data may be in any format and may include patient medical events. Electronic health record data may be viewed as a sequence of medical events. In each medical vent, a timestamp along with one or more medically relevant information segments may be recorded about a patient. The one or more medically relevant information segment values may also be timestamp dependent. Medical event information may include patient demographics such as age, sex, ethnicity, or the like, vital signs, doctor visits and clinical notes, patient reported symptoms, procedures performed, medications prescribed, diagnoses, lab results, imaging data such as radiology, pathology, ultrasound, or the like, bedside monitoring data, genomics data and genetic testing results, billing and coding data, or any other medical information at an individual patient level. Electronic health record data may be structured or unstructured, may be numeric, textual, image, video, or the like, may contain continuous or categorical or binary values, as well as missing values (null), may be stationary or time-varying, or may be a single data item or sequence of data values over time. Electronic health record data may be stored in relational tables in a database schema or other storage.

In some aspects, in order to generate synthetic electronic health records from authentic electronic health records, the authentic health records may be preprocessed and formatted. For example, input authentic electronic health records (e.g., from the authentic electronic health record database 402) may be cleaned and normalized. Cleaning the input electronic health records may include removing medical data that may be inconsistent with valid data (e.g., false alarms, inaccurate data, inconsistent data, or the like). Normalizing the electronic health record data may include normalizing units of measurement, correcting human data entry errors, normalizing drug or procedure codes to a standard dictionary, or the like.

After cleaning and normalizing the authentic electronic health records, each medical event of an electronic health record may be transformed into a numerical vector. For example, A medical event may be represented as a tuple <event-class, event-code, event-value, event-time-stamp>. The event-class may be one of a limited number of possible types of events such as a diagnosis, a lab result, a medication, a procedure, or the like. The event-code may be a category code associated with this event. For example, a procedure, a note, an ICD-10 code (event-class=diagnosis), or a medication (e.g., an NDC code for the medication). The event-value may be a value associated with that event-code. For example, for a medication, the event-value may be a dosage of the medication. For a diagnosis, there may be no associated value, in which case it may be represented as NULL. The event-time-stamp: may be a timestamp when the medical event occurred in actual clock time and date format.

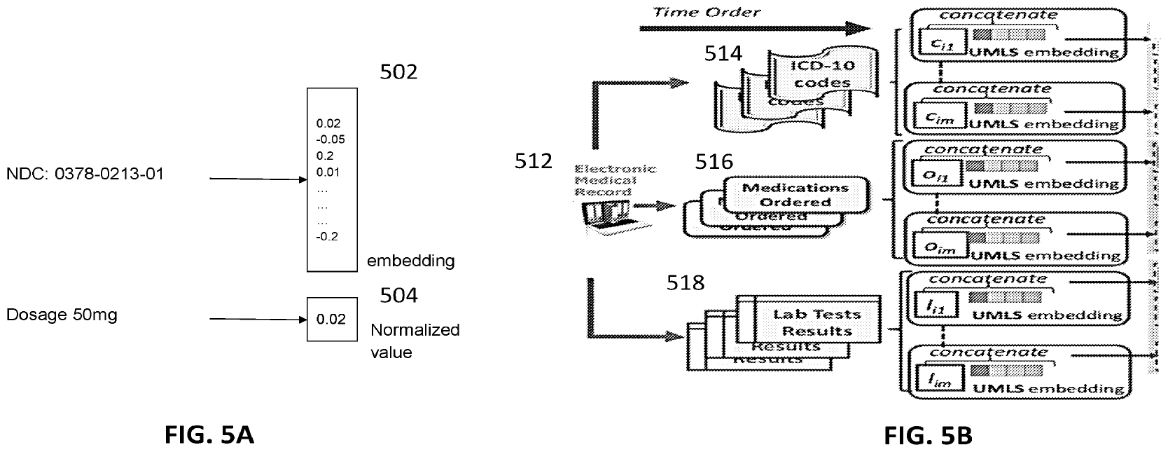

Transforming the electronic health record into a numerical vector may include mapping the event-code to a first vector (of N dimension), normalizing and embedding the event-value (if one exists) into a second vector (of M dimension), and concatenating the first vector and the second vector into a final N+M dimensional vector. For example, if the event-class is medication, and the event-code represents a drug NDC code 0378-0213-01 with an event-value 50 mg (dosage), the representation may include an N-dimensional embedding vector (e.g., vector 502 of FIG. 5A) and a 1-dimensional normalized dosage representation (e.g., vector 504 of FIG. 5A). In this example, the values in the embedding vector may be pulled from the mapping of NDC codes to embedding values, and the dosage value (e.g., 0.02) may be computed as the dosage normalized to be between 0 and 1.

FIG. 5A shows an example transformation of a medical record including the drug NDC code 0378-0213-01 and a dosage of 50 mg. In some implementations, a dictionary that maps event codes to embedding vectors may be pre-computed with random values chosen for the embedding vectors. These embedding vectors may be fixed or mutable during training of the neural network downstream. Pre-computed embeddings may also be used (instead of randomly chosen embeddings) and those values may remain frozen through any subsequent training of the neural network; for example the embeddings generated offline from a unified medical language system (UMLS).

FIG. 5B depicts how unified medical language system (UMLS) embeddings may be generated. As shown in the example of FIG. 5B, and electronic medical record 512 may include an international statistical classification of disease and related health problems (ICD) code 514, medications ordered 516, and lab test results 518 over time. These medical events of the medical record 512 may be transformed into the concatenated vector 520 using embedding vectors and methods described above.

After these pre-processing steps, the electronic health record for each individual patient (e.g., authentic electronic health records stored in database 402) may be represented as a sequence of medical events, each event represented as a numeric vector. These transformed electronic health records may be inputted into the generative model 405 or 305 for training the model 305 or 405. In some aspects, the training engine 110, the generative model 305, and/or the generative model 405 includes a generative adversarial model (GAN) architecture which may include at least two neural networks such as a generator network and a discriminator network. The generator network may randomly generate synthetic data that is meant to look as close as possible to “fake” real data. For example, generate synthetic electronic health records (e.g., synthetic health information 325, synthetic electronic health records data 425, or the like) that resemble the authentic electronic health records stored in database 402. The discriminator network (e.g., discriminator 610) may learn to determine whether a given data record is from the generator model distribution or the real/authentic data distribution, and sends feedback (e.g., in the form of gradient updates) to the generator so it may improve its generation of synthetic data, and to the discriminator so it can improve its ability to detect fake records.

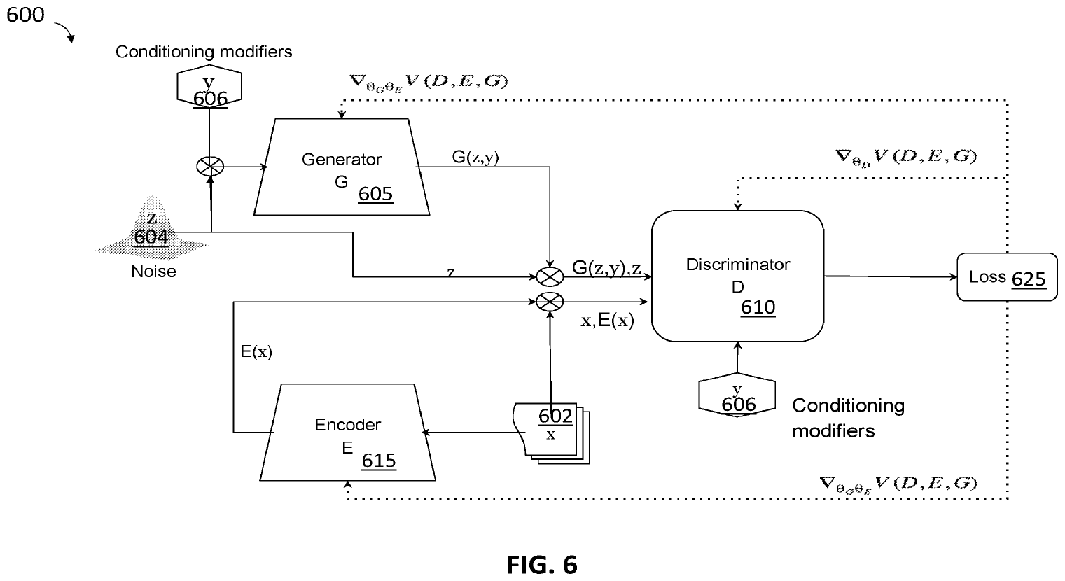

FIG. 6 depicts training an example of a generative adversarial network (GAN) 600, in accordance with some example embodiments. As shown in the example of FIG. 6, the GAN 600 includes a generator 605, a noise generator 604, a conditioning modifier 606, a discriminator 610, and an encoder 615 receiving authentic electronic medical events x 602, and a loss distribution 625. As further shown, the generator 605 may receive inputs of conditioning modifiers 606 and noise samples from the noise generator 604 and may output synthetic electronic medical records G(z,y), where z represents random noise and y represents a conditioning variable. The output synthetic electronic medical records G(z,y) and the authentic electronic medical events x 602 may be inputted to the discriminator 610. G(z,y) may be outputted as a sequence of numeric vectors, each numerical vector may represent a medical event. The discriminator 610 may be configured to detect fake (e.g., synthetic) samples by, for example, determining a distribution over the state of the sample (e.g., real or fake) based on the received inputs. The determined distribution may be represented as the loss distribution 625 which may indicate whether an analyzed sample is authentic or synthetic. As further shown in the example of FIG. 6, the loss distribution 625 may be fed back to the generator 605, the discriminator 610, and/or the encoder 615 to update each with the new loss distribution 625 data to improve the function of the generator 605, the discriminator 610, and/or the encoder 615.

In some aspects, the generator 605, the discriminator 610, and/or the encoder 615 may be trained using the authentic medical event 602, noise samples from the noise generator 604, and/or the conditioning modifiers 606. For example, the generator 605, the discriminator 610, and/or the encoder 615 may undergo multiple of training iterations. In one example iteration, the system 600 may sample a batch for m noise samples from the noise generator 604 {z⁽¹⁾, . . . , z^{(m)}} from prior noise p _{G}(z), and their associated label y⁽ⁱ⁾ if using conditioning. Next, the system 600 may sample a batch of m examples of authentic electronic medical events x 602 {x⁽¹⁾, . . . , x^{(m)}} from data generating distribution p _{Data}(x), the encoder 615 may compute their encoded form E(x) for each i, and their associated label y⁽ⁱ⁾ (e.g., conditioning variable) if using conditioning modifiers 606. The system 600 may then update the discriminator 610 by ascending its stochastic gradient

$${∇}_{{θ}_{G}} \frac{1}{m} {\sum }_{i = 1}^{m} log ( 1 - D ( G ( {z}^{( i )} , {y}^{( i )} ) , {y}^{( i )} ) .$$

Additionally, the system 600 may sample a batch of m noise samples from the noise generator 604 {z⁽¹⁾, . . . , z^{(m)}} from prior noise p _{G}(z), and their associated label y⁽ⁱ⁾ (e.g., conditioning variable) if using conditioning modifier 606. The system 600 may then update the generator 605 and the encoder 615 by descending its stochastic gradient:

$${∇}_{{θ}_{G}} \frac{1}{m} {\sum }_{i = 1}^{m} log ( 1 - D ( G ( {z}^{( i )} , {y}^{( i )} ) , {y}^{( i )} )$$

Various embodiments may utilize different architectures for the encoder 615 and the generator 605. One example embodiment of the encoder 615/generator 605 pair may utilize a variant of a RNN (Recurrent Neural Network) with LSTM (long short-term memory). The RNN may also use a gated recurrent unit (GRU) cells instead of a LSTM. LSTM (or GRU) is a neural network architecture that has feedback connections and allows the neural network to process entire sequences of data such as speech, video, or time-based data (e.g., electronic medical records). LSTM (or GRU) however may have an implicit assumption of uniformly distributed time-steps, whereas with medical events, it may be the case that a single patient's medical event distribution in time is highly non-uniform as the gap between events can be in hours, days or even years. The generator 605 may utilize implicit health information in the spacing of events: close together events may imply the patient is in or near to an acute illness, whereas events are more likely to be spaced well apart when the patient is healthy.

The system 600 may utilize a T-LSTM (time-aware LSTM) cell (instead of a standard LSTM cell) to capture the time component and sequential nature of the data. The T-LSTM may be configured to handle irregular time intervals in longitudinal patient records. The encoder 615 and generator 605 may form an auto-encoder-like pair. The hidden states of the T-LSTM encoder may be a sequence of intermediate outputs hₜ representing a patient-state at various points in time as the sequence of patient medical events is processed by the encoder 615. h_{T}, the hidden state at the last encoder timestamp T, may include a single compact representation of the entire sequence of medical events for that patient, which may be referred to as a patient-state. The RNN architecture may include a decoder (not shown) that may be configured to then take any vector in a patient-space (e.g., a vector from the encoder 615) and transform it back into a sequence of numeric vectors representing medical events.

The encoder 615/generator 605 pair may also be based on a transformer architecture. Unlike the RNN architecture described above, where the sequence of medical events is consumed in sequential time order by the neural network, the transformer architecture may look at the entire sequence of medical events together in a single layer (e.g., no recurrence), adding an “attention” mechanism to allow the network to efficiently model dependencies between different medical events in the sequence.

The transformer architecture's encoder may include a stack of N encoders (e.g., typically N=6 is used), and similarly the transformer architecture may include a stack of N decoders/generators. Since the transformer may not process the sequence one item at a time, the transformer may utilize positional encoding to allow the network to learn positional interaction of events in the sequence. The positional encoding may be a calculated numeric vector PE(t), where t is the position in the sequence. Then the value of PE(t) may be numerically added to the input data in each time step.

The generator 605 may be designed with the ability to generate the sequence of medical events from the encoded representation (e.g., the output of the encoder 615).

Some implementations may utilize a federated learning architecture, allowing the system (e.g. system 600) to train the generative model (e.g. generator 605) using one or more training datasets, distributed across one or more physical locations (e.g. other hospital systems or other campuses).

In some implementations of machine learning where security and privacy may not be of highest concern, if multiple datasets are available, they may be copied to a single location and merged to form a single unified dataset. In some aspects, the system (e.g., system 600) may utilize federated learning to allow training of the generative model 605 without the need to copy datasets to a single location, thus removing potential legal and/or regulatory burdens associated with such data sharing requirements, as well as dramatically reducing an amount of data that may be transmitted from one location to another.

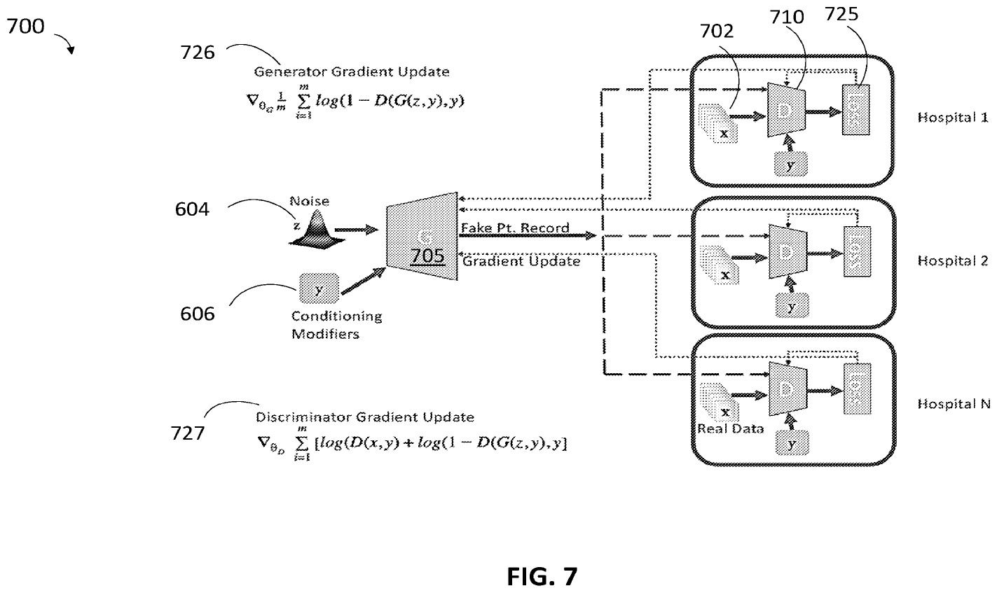

FIG. 7 depicts training an example generative adversarial network 700 using a federated learning structure, in accordance with some example embodiments. As shown in the example of FIG. 7, a central generator network 705 generates synthetic electronic medical records based on the noise generator 604 and conditioning modifiers 606 (if any). The output of the generator 705 may be input to one or more discriminator networks 710 of different entities (e.g., hospitals) which discriminate between the generated synthetic electronic medical records and the authentic electronic medical records 702. The one or more discriminator networks 710 may output a loss distribution 725 which may be fed back to the generator 705 and the one or more discriminators 710 to update a gradient of the generator 705 and/or the one or more discriminators 710 to improve the generation of synthetic data (e.g., output of the generator 705) or improve the discrimination of the synthetic data versus the authentic data 702 (e.g., loss 725). As further shown in FIG. 7, the authentic medical records 702 are not transmitted between the different medical entities (e.g., hospitals 1-N) which may reduce or eliminate unintended sharing of confidential patient electronic medical records.

In some aspects, the generator 705 may send batches of generated patient records to each of the discriminator networks 710 (one in each location or hospital). Each discriminator 710 may randomly select a batch of real patient data from its local repository, and may run the discriminator function against the data sent from the generator 705. Based on the loss distribution 725 of the discriminator 710, a hospital server may calculate a gradient update 727 for the discriminators 710, and may update its local discriminator 710 based on the loss 725. The hospital server may also update its local encoder (not shown) with an appropriate gradient update for the encoder. The server may then calculate a gradient update 726 for the generator 705, and may send those values back to the generator 705. The generator 705 may update its generator model by aggregating the updates from all hospital servers (discriminators 710) and may now be ready for generating new fake data in an attempt to fool the discriminators 710 in a next iteration.

After the generative model (e.g., generative model of generator 305, 405, 605, and 705) is trained, a synthetic electronic medical record system (e.g., system 300, 400, 600, and 700) may utilize the trained generator to generate patient records for a synthetic dataset, based on requirements (e.g., conditioning modifiers 306 or 606) provided by an end user via a graphical user interface or representational state transfer (REST) application programming interface (API) (e.g., of the front end system 410). In some implementations, modifiers or post-generation filtering may be used to further condition the generated patient data so that it is consistent with certain desired conditions (conditioning modifiers 306 or 606).

Generated synthetic medical records may be validated to ensure quality of the synthetic medical record. Many different types of validation may be used such as validation of electronic health record quality and validation that the generated electronic health records do not leak any real patient health information data from the training set.

In order to validate electronic health record data, a visual inspection of the synthetic medical record itself may not be sufficient. A set of mechanisms may be employed to help with this validation process, including statistical validation, comparative predictive modeling, expert clinical review. For statistical validation, the generative system may utilize statistical measures over a population to demonstrate that the generated data has similar characteristics to the original/authentic data. For example, the system may utilize a distribution visualization called a violin plot to determine whether the synthetic data is comparable to the authentic data within an acceptable threshold. The system may also utilize other statistical validation such as a plot of a univariate correlation between each pair of variables in the original authentic electronic health record dataset and compare this against the same correlation plot in the synthetic electronic health record dataset.

For comparative predictive modeling, the system may select a set of N specific real-world predictive problems (such as hospital readmission, diagnosis prediction, length of stay prediction, or the like). The system may run a variety of tests for the predictive problems such as training the generative model with the synthetic data and predicting with the real data and training the model with the real data and predicting with the synthetic data. The predictive accuracy of both approaches may then be compared and the synthetic dataset may be assumed validated if a certain threshold of confidence between a relevant metric of predictive performance is achieved. A variety of relevant metrics may be used such as a receiver operating characteristic area under the curve (ROCAUC) metric for model performance or a standard z-test with a 95% confidence interval.

For expert review, a random subset of synthetically generated patient data may be selected for validation. This data may be sent to expert reviewers which have medical expertise (e.g. trained as a physician) to validate the quality of the randomly selected sample of synthetic data.

For validation that the generated electronic health records do not leak any real patient health information data from the training set, the system may use a combination of dynamic time warping, as well as other relevant matching functions to define a distance metric, such as a high-dimensional cosine distance, between any two electronic health records (regardless of whether they are real or synthetic), and compare each possible pair of real record to synthetic records. After comparing, if any of the pairs have a distance value that is too low or within a threshold distance, the system may highlight that pair for manual review by a human to determine if any authentic patient medical data has leaked into the generated synthetic data. It should be noted that the synthetic electronic health records are generated from random noise (e.g., random noise generator 304, 604) as an input of the generator (e.g., generator 305, 405, 605, or 705) and may not be likely to include any real electronic health record data.

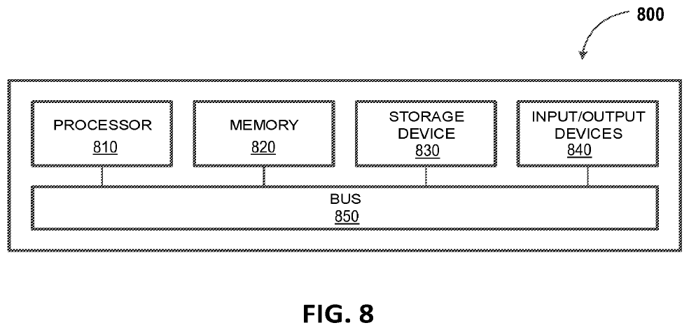

FIG. 8 depicts a block diagram illustrating a computing system 800 consistent with implementations of the current subject matter. Referring to FIGS. 1-7, the computing system 800 can be used to implement the training engine 110, the neural network engine 140, the client device 130, the backend system 450, the generator 305, 405, 605, 705, the discriminator 610, 710, the encoder 615, and/or any components therein.

As shown in FIG. 8, the computing system 800 can include a processor 810, a memory 820, a storage device 830, and input/output devices 840. The processor 810, the memory 820, the storage device 830, and the input/output devices 840 can be interconnected via a system bus 850. The processor 810 is capable of processing instructions for execution within the computing system 800. Such executed instructions can implement one or more components of, for example, the machine learning controller 110. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a solid state drive, a floppy disk device, a hard disk device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

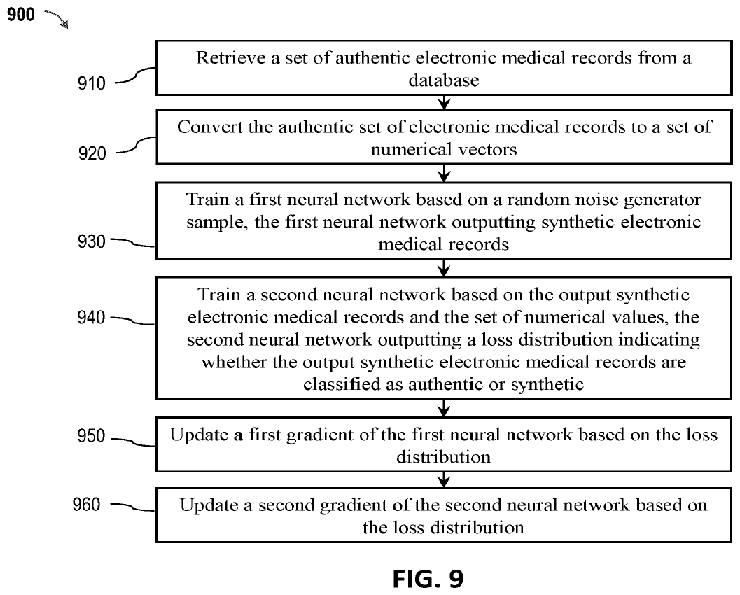

FIG. 9 depicts a flowchart illustrating an example of a process 900 for generating synthetic patient data, in accordance with some example embodiments. Referring to FIGS. 1-8, the process 900 may be performed by a computing apparatus such as, for example, the training engine 110, the neural network engine 140, the client device 130, the generator 305, 405, 605, 705, the discriminator 610, 710, the encoder 615, a server, the computing system 800 and/or the like.

At operational block 910, the training engine 110 may retrieve a set of authentic electronic medical records from a database (e.g., database 302 or 402). In some aspects, the training engine 110 may retrieve the set of authentic electronic medical records in response to receiving a request to generate synthetic electronic medical records from a front end system (e.g., front end system 410).

At operational block 920, the training engine 110 may convert the authentic set of electronic medical records to a set of numerical vectors. For example and with reference to FIG. 6, the encoder 615 may receive the authentic set of electronic medical records 602 and apply an embedding vector (e.g., embedding vector 502). The embedding vector may map a medical event code to a first vector. The encoder 615 may also normalize and embed a medical event value into a second vector. The encoder 615 may also concatenate the first vector and the second vector into a final vector of the set of numerical vectors.

At operational block 930, the training engine 110 may train a first neural network based on a random noise generator sample. The first neural network may output synthetic electronic medical records. For example, the noise generator 304 or 604 may provide the sample of random noise to the generator 605, 705. The output synthetic electronic medical records may be in a numerical vector format.

At operational block 940, the training engine 110 may train a second neural network using the output synthetic electronic medical records and a set of numerical values. The second neural network may output a loss distribution indicating whether the output synthetic electronic medical records are classified as authentic or synthetic.

At operational block 950, the training engine 110 may update a first gradient of the first neural network based on the loss distribution. For example, the updating may include descending the first gradient. The first gradient may include

$${∇}_{{θ}_{G}} \frac{1}{m} {\sum }_{i = 1}^{m} log ( 1 - D ( G ( {z}^{( i )} , {y}^{( i )} ) , {y}^{( i )} ) .$$

At operational block 960, the training engine 110 may update a second gradient of the second neural network based on the loss distribution. For example, updating the second gradient may include ascending the second gradient. The second gradient may include

$${∇}_{{θ}_{D}} \frac{1}{m} {\sum }_{i = 1}^{m} [ log ( D ( {x}^{( i )} , {y}^{( i )} , {E ( x )}^{( i )} ) + log ( 1 - D ( G ( {z}^{( i )} , {y}^{( i )} ) , {y}^{( i )} ) ] .$$

In some aspects, updating the first gradient or updating the second gradient may continue until the loss distribution satisfies a threshold. The threshold may indicate that the first neural network and/or the second neural network have been sufficiently trained to either generate the synthetic electronic medical records or discriminate between the generated synthetic electronic medical records and the authentic electronic medical records.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions which, when executed by the at least one data processor, result in operations comprising:
retrieving a set of authentic electronic medical records from a database;
converting the authentic set of electronic medical records to a set of numerical vectors;
training a first neural network based on a random noise generator sample, the first neural network outputting synthetic electronic medical records; and
training, based on the output synthetic electronic medical records and the set of numerical vectors, a second neural network, the second neural network outputting a loss distribution, the loss distribution indicating whether the output synthetic electronic medical records are classified as authentic or synthetic,
wherein training the first neural network further comprises updating a first gradient of the first neural network based on the loss distribution, wherein training the second neural network further comprises updating a second gradient of the second neural network based on the loss distribution.

2. The system of claim 1, wherein training the first neural network further comprises receiving a conditioning modifier, the conditioning modifier altering at least one characteristic of the synthetic electronic medical records.

3. The system of claim 2, wherein receiving the conditioning modifier comprises receiving the conditioning modifier via a user interface.

4. The system of claim 1, wherein training the first neural network is in response to receiving a request for synthetic electronic health records from a front end system.

5. The system of claim 1, wherein updating the first gradient comprises descending the first gradient.

6. The system of claim 4, wherein the first gradient comprises
$${∇}_{{θ}_{G}} \frac{1}{m} {\sum }_{i = 1}^{m} log ( 1 - D ( G ( {z}^{( i )} , {y}^{( i )} ) , {y}^{( i )} )$$

7. The system of claim 1, wherein updating the second gradient comprises ascending the second gradient.

8. The system of claim 6, wherein the second gradient comprises
$${∇}_{{θ}_{D}} \frac{1}{m} {\sum }_{i = 1}^{m} [ log ( D ( {x}^{( i )} , {y}^{( i )} , {E ( x )}^{( i )} ) + log ( 1 - D ( G ( {z}^{( i )} , {y}^{( i )} ) , {y}^{( i )} ) ] .$$

9. The system of claim 1, wherein the first neural network comprises a recurrent neural network.

10. The system of claim 8, wherein the recurrent neural network utilizes a time aware long short term memory.

11. The system of claim 8, wherein the recurrent neural network utilizes a gated recurrent unit.

12. The system of claim 1, wherein the operations further comprise:
validating the synthetic medical records, wherein the validating comprises comparing a statistical distribution of the synthetic medical records to a statistical distribution of the authentic medical records.

13. The system of claim 11, wherein the validating further comprises comparing a predictive model performance of the synthetic medical records to a predictive model performance of the authentic medical records.

14. The system of claim 1, wherein the second neural network is distributed across multiple devices in separate locations in a federated learning structure.

15. A computer-implemented method, comprising:
retrieving, by a processor, a set of authentic electronic medical records from a database;
converting, by an encoder, the authentic set of electronic medical records to a set of numerical vectors;
training, by the processor, a first neural network based on a random noise generator sample, the first neural network outputting synthetic electronic medical records; and
training, by the processor, a second neural network using the output synthetic electronic medical records and the set of numerical vectors, the second neural network outputting a loss distribution indicating whether the output synthetic electronic medical records are classified as authentic or synthetic,
wherein training the first neural network comprises updating a first gradient of the first neural network based on the loss distribution, wherein training the second neural network comprises updating a second gradient of the second neural network based on the loss distribution.

16. The method of claim 14, wherein training the first neural network further comprises receiving a conditioning modifier, the conditioning modifier altering at least one characteristic of the synthetic electronic medical records.

17. The method of claim 15, wherein receiving the conditioning modifier comprises receiving the conditioning modifier via a user interface.

18. The method of claim 14, wherein training the first neural network is in response to receiving a request for synthetic electronic health records from a front end system.

19. A non-transitory computer readable medium storing instructions, which when executed by at least one data processor, result in operations comprising:
retrieving a set of authentic electronic medical records from a database;
converting the authentic set of electronic medical records to a set of numerical vectors;
training a first neural network based on a random noise generator sample, the first neural network outputting synthetic electronic medical records; and
training a second neural network using the output synthetic electronic medical records and the set of numerical vectors, the second neural network outputting a loss distribution indicating whether the output synthetic electronic medical records are classified as authentic or synthetic,
wherein training the first neural network comprises updating a first gradient of the first neural network based on the loss distribution, wherein training the second neural network comprises updating a second gradient of the second neural network based on the loss distribution.
